(19) United States
(12) Patent Application Publication
(10) Pub. No.: US 2019/0268574 A1
(43) Pub. Date: Aug. 29, 2019

(54) COMMUNICATION SYSTEM FOR A RETRACTOR WITH A MOUNTED CAMERA

(71) Applicant: Rebound Therapeutics Corporation, Irvine, CA (US)

(72) Inventors: Ross Tsukashima, Irvine, CA (US); Peter G. Davis, Irvine, CA (US)

(73) Assignee: Rebound Therapeutics Corporation, Irvine, CA (US)

(21) Appl. No.: 15/908,608

(22) Filed: Feb. 28, 2018

(51) Int. Cl. H04N 7/18; H04N 5/225; H04N 7/10; A61B 17/02; A61B 1/313; A61B 1/00; A61B 1/05; A61B 1/045; A61B 1/06; A61B 5/00

(52) U.S. Cl. CPC H04N 7/185; H04N 5/23216; H04N 7/10; A61B 17/0218; A61B 1/3132; A61B 1/00009; A61B 1/00018; A61B 1/051; A61B 1/045; A61B 1/00006; A61B 1/00114; A61B 1/00029; A61B 1/0676; A61B 5/6847; A61B 1/00045; H04N 2005/2255; H04N 5/2256

(57) ABSTRACT

A communications system for video data for a retractor with a proximally mounted camera.

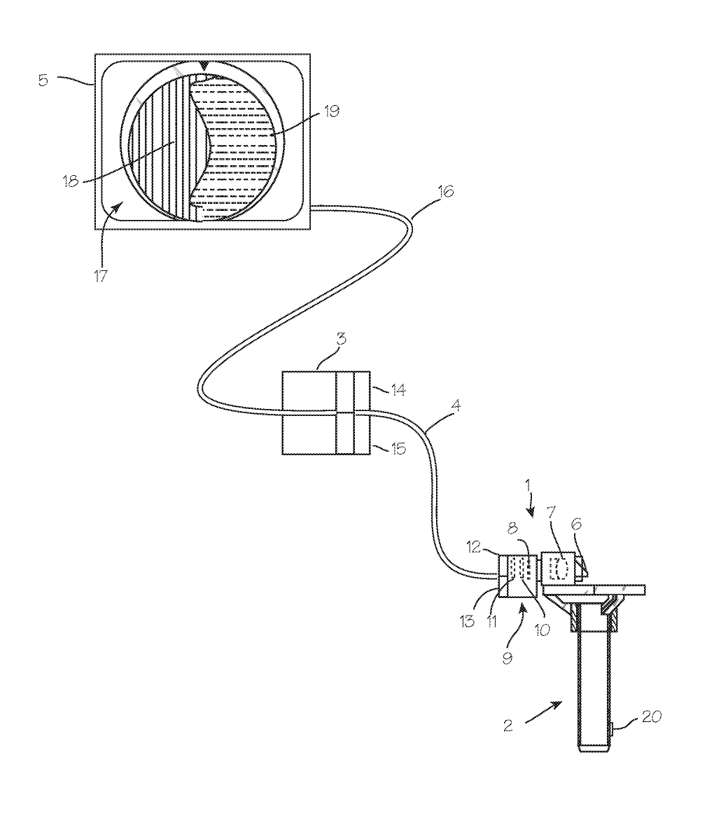

### FIELD OF THE INVENTIONS

The inventions describe below relate to the field of imaging for minimally invasive surgery.

### BACKGROUND OF THE INVENTIONS

Many minimally invasive surgeries may be facilitated by the use of cameras to obtain video of a small surgical space while a surgeon is using tools within the surgical space. Instead of looking directly at the surgical space through narrow minimally invasive access ports, a surgeon can look at an enlarged image of the surgical space on a display. Especially when working in the brain, high definition, low lag time, and high magnification are helpful to ensure that the surgeon can see and operate properly within the surgical field (for example, to ensure that the surgeon excises unwanted tissue and leaves healthy tissue intact). Surgeons are accustomed to high definition (720 p, for example) in video displays used for visualizing surgery.

When using cameras close to the surgical field, this has required use of HDMI cables between the camera and an associated control box. In our prior U.S. patent application Ser. No. 15/576,536 (Published in the PCT as Cannula With Proximally Mounted Camera, WO/US2018/035366 (Feb. 22, 2018) we disclose cannulas with proximally mounted cameras for use in minimally invasive surgery in the brain and elsewhere in the body. To provide high definition imaged from the camera, an HDMI cable has been necessary to connect the proximally mounted camera to a control box, as the HDMI standard is the standard for transmission of high definition video. HDMI cables have numerous parallel conductors (an HDMI cable contains four shielded twisted pairs and seven separate conductors, for a total of 15 conductors) which are considered necessary to transmission of the large high definition video data stream. The imaging device of the camera provides video data in a parallel video data stream for faster transfer of high resolution image data.

HDMI cables are bulky and stiff, and make it difficult to keep a retractor in place during surgery, and make it difficult to adjust the position of a retractor with a proximally mounted camera. In brain surgery in particular, tending to the positioning and constant re-positioning of a retractor due to the movement caused by the bulk of a video cable should be avoided so the surgeon can concentrate on the procedure being performed.

### SUMMARY OF THE INVENTIONS

The inventions described below provide for improved visualization and less disruption arising from dislodgement of a retractor and camera system during minimally invasive surgery, and easier placement and manipulation of retractors used to provide access to a minimally invasive surgical space. The new system includes a retractor and an imaging system for minimally invasive surgery which includes a surgical retractor, a camera assembly disposed on the retractor, a control box for processing a high definition video data stream from the camera (and transmission to a display), and a single low-profile cable communicating from the camera assembly to the control box. The system dispenses with the bulky HDMI cable previously needed for obtaining high resolution images from a high resolution video camera, and uses a single-conductor cable such as a coaxial cable (when only the center conductor is used) or a two-conductor cable such as a twisted-pair cable, or a coaxial cable when both the central conductor and conducting shield are used, to transmit a high resolution video data stream from the camera to the control box, and to transmit control signals from the control box to the camera. This is made possible by incorporating a serializer within the camera assembly to serialize video data from an image sensor, and transmit serialized video data to the control box, and incorporating a de-serializer into the control box to de-serialize serialized video data received from the camera assembly and transmit de-serialized data to a display. The high-resolution video may be standard high definition video, or other high-resolution video. The system can also use the cable to pass signals corresponding to status of the camera, or to pass signals from sensors on the retractor to the control box, or to pass camera control signals from the control box to the camera assembly to control operation of the camera assembly. Camera control signals can include signals to control image processing parameters (contrast, brightness, orientation, etc.) or camera operating parameter (focus, aperture, etc.). Video processing tasks, such as adjusting contrast, brightness, orientation, etc. of the image are performed in the camera assembly, in an embedded controller, rather than in image processors in the control box, to limit lag time between image acquisition and image display. The system may also provide power to the camera through the cable.

### BRIEF DESCRIPTION OF THE DRAWINGS

The FIGURE is a block diagram of the communications system for a retractor with proximally mounted camera.

### DETAILED DESCRIPTION

The FIGURE is a block diagram of the communications system for a retractor with camera mounted on, or proximate, the retractor. A camera assembly 1, or the image sensing and optical components of a camera, are disposed on the retractor 2. The camera is connected to a control box 3 through a cable 4, and the control box is connected to a display screen 5 The camera assembly 1 comprises the prism or reflector (or set of mirrors) 6, a lens or lenses 7, the imaging device 8 and the control system 9 (provided in the camera component of the system). The lens 7 may be part of an optical assembly that includes additional optical components (filters, beam splitters, etc.). The imaging device 8 may be any suitable image sensor such as a CCD sensor or CMOS sensor. The imaging device provides video data in a parallel video data stream. The control system 9 includes an embedded controller 10, a transmitter and receiver 11 and appropriate image processing software to control the camera. The control system can be integrated into the image sensor, or it may be an embedded controller/logic device such as a microprocessor or Field Programmable Gate Array (FPGA). The transmitter and receiver comprise a serializer 12, operable to receive parallel image data from the image sensor, convert it to serial image data, and transmit it to the control box. The transmitter and receiver may also comprise a de-serializer 13, operable to receive serial control data from the control box, convert it to parallel data, and transmit it to the embedded controller (in formats such as I2C or SPI). The serializer and de-serializer may be combined into a single component (a serializer/de-serializer), though the de-serializer function might not be used for control signals transmitted from the control box. The image sensor could be controlled directly by the serializer/deserializer. The embedded controller could be on the deserializer side of the serializer/deserializer in the camera assembly, controlling the image sensor.

(A serializer and de-serializer is an integrated circuit transceiver that converts parallel data to serial data and vice-versa. A serializer is an integrated circuit that converts parallel data to serial data, and a de-serializer is an integrated circuit that converts serial data to parallel data. They may be packaged together, in which case the combined serializer/de-serializer is operable as a transceiver to both receive a parallel data stream and convert it to a serial data stream, and receive a serial data stream and convert it to a parallel data stream. In the system shown in the FIGURE, the primary emphasis is in transmitting the high volume video data stream from the camera assembly to the control box (and further on to the display), so that a serializer may be used (without a de-serializer) in the camera assembly, and a de-serializer may be used (without a serializer) in the control box, though a combined serializer/de-serializer may be installed at each end of the system, and all data streams going to and from the control box, and to and from the camera assembly, may be serialized for transmission.)

The cable 4 connects the camera assembly to the control box 3. The cable is a coaxial cable or a twisted pair cable. Preferably, the cable has no more than two conductors for carrying the video data stream. The cable may be a coaxial cable with a single center conductor and a conducting shield, without additional conductors (an RG174 type coax cable, less than 3 mm in outer diameter, for example), or it may be a twisted pair of conductors, also without additional conductors. For the coaxial cable, only the center conductor may be used to transmit signals, while the conducting shield together with the center conductor are used to transmit power, or both the single center conductor and the conducting shield may be used to transmit data, while no additional conductors are used to transmit data.

The control box 3 includes a de-serializer 14 operable to receive serial image data from the camera serializer, convert it to parallel image data, and transmit it to the display. The transmitter may also comprise a serializer 15, operable to receive parallel control data from the control box, convert it to serial data, and transmit it to the embedded controller (through the de-serializer 13 in the camera assembly). The de-serializer 14 and serializer 15 may be combined into a single component (a serializer/de-serializer), though the de-serializer function might not be used for control signals transmitted from the control box. The control box transmits video data through any suitable cable 16 and/or interface to the display 5. The display is operable to present a video image 17 of the surgical field. The surgical field could, for example, be a region of the brain of a patient with a blood mass 18 that must be removed, and healthy brain tissue 19.

The de-serializer is operable to de-serialize serialized video data received from the camera assembly and transmit de-serialized data to the display. The control box can also include circuitry and programming making it operable to generate control signals, in response to user input or automatic image analysis, and transmit those control signals to the embedded controller in the camera assembly. Control signals can include signals to adjust camera parameters (such as focus and aperture) and signals to adjust image parameters of the image data generated by the image sensor and embedded controller (such as image brightness, image saturation, image orientation). Performing image adjustments in the embedded controller, rather than in the control box, is advantageous because it reduces perceptible lag time between acquisition of the image by the image sensor and eventual display of the image on the display.

As described above, the video data stream undergoes several transformations en route from the image sensor to the display screen. The image sensor generates video data in a first parallel video data stream, the serializer in the camera converts the first parallel video data stream to a serial data stream, then the de-serializer in the control box converts the serial data stream to a second parallel video data stream for output to a display. Video data transmitted to the display may be transmitted at a different data rate than video data received by the control box from the serializer.

The control box may be operable for additional functions. For example, the control box may provide power for the camera and control system, over the cable, so that only a single cable must be used to support the camera operation. Likewise, lights (LED's) may be provided in the retractor, to illuminate a surgical field within the surgical space, and the control box may provide power to the lights, again through the single cable. The control box and coaxial cable can be configured so that the control box is operable to transmit power through the conducting shield of the coaxial cable to the camera or the lights. The control box can include user inputs to accept input from an operator to energize the lights, adjust the brightness, color temperature or color of the lights, and generate and transmit corresponding control signals to a lighting controller (which may be in the control box or the camera assembly or elsewhere) and transmit power through the cable to the lights.

The retractor may be any type of retractor, such as a cannula tube or a bladed retractor. The FIGURE illustrates a cannula tube, suitable for use in brain surgery and spine surgery. The system can be used for a variety of minimally invasive surgeries or inspections, including brain surgery, spinal surgery, endoscopic surgery, arthroscopic surgery, ear, nose and throat inspections and procedures, and others. The FIGURE depicts the camera at the proximal end of the retractor, but it may be positioned at the distal end of the retractor, or, for a blade retractor typically used for spine surgery, anywhere along the length of the retractor, so long as a viewing axis of the camera assembly is directed toward the surgical field.

Various sensors 20 can be included on the retractor, on or integrated into the retractor body. For example, force sensors, pH sensors, impedance sensors, temperature sensors, glucose sensors or position sensors (gyros or accelerometers) can be disposed proximate the distal or proximal end of the retractor to detect various biological parameters or other parameters. Corresponding signals received by the control system, embedded controller or other processor and can be transmitted through the cable from the embedded controller or other processor to a control box operable to display and/or record data.

While the system is illustrated with a single camera mounted on a single retractor, the system may be implemented with two or more cameras on a single retractor, or cameras on multiple retractors when used together to support a surgery.

While the preferred embodiments of the devices and methods have been described in reference to the environment in which they were developed, they are merely illustrative of the principles of the inventions. The elements of the various embodiments may be incorporated into each of the other species to obtain the benefits of those elements in combination with such other species, and the various beneficial features may be employed in embodiments alone or in combination with each other. Other embodiments and configurations may be devised without departing from the spirit of the inventions and the scope of the appended claims.

We claim:

1. A retractor and imaging system for minimally invasive surgery, said system comprising:
a surgical retractor, said surgical retractor having a distal end adapted for insertion into the body of a patient and a proximal end which, during use, remains outside the body of the patient;
a camera assembly disposed on the retractor, said camera assembly comprising an image sensor, an embedded controller and a serializer, said camera assembly operable to generate video data, serialize the video data, and transmit serialized video data;
a control box operable to receive video data from the camera;
said control box comprising a de-serializer operable to de-serialize serialized video data received from the camera assembly and transmit de-serialized data to a display; and
a cable connecting the control box and the camera assembly, with said camera assembly and control box, said cable operable to pass the serialized data from the serializer to the de-serializer.

2. The system of claim 1, wherein:
the control box is further operable to generate camera control signals and transmit said camera control signals to the embedded controller of the camera assembly, and
the embedded controller is further operable to control camera parameters and image processing parameters in response to camera control signals; and
the cable is further operable to pass camera control signals from the control box to the embedded controller of the camera assembly.

3. The system of claim 1, wherein the cable is a coaxial cable.

4. The system of claim 1, wherein the cable is a twisted pair cable having only a single pair of conductors.

5. The system of claim 3, wherein the coaxial cable has an outer diameter of less than 10 mm.

6. The system of claim 3, wherein the coaxial cable has an outer diameter of less than 3 mm.

7. The system of claim 3, wherein the coaxial cable is the only cable communicating between the control box and the camera assembly.

8. The system of claim 1, wherein the control box is further operable to supply power to the camera through the cable.

9. The system of claim 1, wherein the cable is further operable to supply power to the camera from a power supply.

10. The system of claim 1, further comprising a light disposed on the retractor for illuminating a surgical field in the body, wherein the control box is further operable to supply power to the light through the cable.

11. The system of claim 1, further comprising a biological parameter sensor disposed on the retractor, wherein the control box is further operable to supply power to the biological parameter sensor.

12. The system of claim 1, wherein the embedded controller is further operable to generate signals corresponding to a status of the camera assembly and transmit signals corresponding to the status of the camera assembly, along with the serialized image data, to the control box through the coaxial cable.

13. The system of claim 3, wherein the coaxial cable comprises a single center conductor and a conducting shield.

14. The system of claim 1, wherein the image sensor is operable to generate parallel video data, the serializer is operable to convert the parallel video data to serial video data, the de-serializer is operable to convert the serial video data to parallel video data, and the control box is operable to transmit parallel video data to a display.

15. The system of claim 1, further comprising sensors integrated into the retractor, operably connected with a control system, and said control system is operable to receive signals from said sensors and transmit them through the serializer and cable to the control box.

16. The system of claim 1, wherein the image sensor is operable to generate video data in a first parallel video data stream, the serializer is operable to convert the first parallel video data stream to a serial data stream, the de-serializer is operable to convert the serial data stream to a second parallel video data stream for output to the display.
